(19) United States
(12) Patent Application Publication
(10) Pub. No.: US 2003/0087429 A1
(43) Pub. Date: May 8, 2003

(54) Dendritic cells isolation methods

(75) Inventor: Jeffrey G. Moore, Kennebunkport, ME (US)

Correspondence Address: HALE AND DORR, LLP, 60 STATE STREET, BOSTON, MA, 02109

(73) Assignee: Phylogix, Inc., Scarborough, ME (US)

(21) Appl. No.: 10/190,258

(22) Filed: Jul. 3, 2002

Related U.S. Application Data
(60) Provisional application No. 60/303,265, filed on Jul. 5, 2001.

(51) Int. Cl. C12N 5/06; C12N 5/08

(52) U.S. Cl. 435/325; 435/370; 435/372

(57) ABSTRACT

Disclosed are methods for isolating dendritic cells and/or dendritic progenitor cells. The methods include contacting a population of cells with a plurality of FRIL family member molecules, and removing the unbound cells, wherein the cells bound to the FRIL family member molecules are an isolated population of dendritic cells and/or dendritic progenitor cells.

### CROSS REFERENCE TO RELATED APPLICATION

This application claims benefit from U.S. Provisional Application Serial No. 60/303,265 filed Jul. 5, 2001, the entire contents of each of which are hereby incorporated by reference.

### BACKGROUND OF THE INVENTION

The invention relates to the field of dendritic cells, dendritic progenitor cells, and methods for isolating these cells.

The body's immune system clears foreign antigens through a complex series of steps involving several types of bone marrow-derived immune cells. Antigens first come in contact with the immune system by interacting with dendritic cells (DCs) located in skin and mucosal membranes. Dendritic cells (DCs) are a rare type of antigen presenting cell that originate in the bone marrow and can be found distributed throughout the body (Steinman, Annu. Rev. Immunol. 9: 271-296, 1991). Inflammation resulting from infection attracts large numbers of additional DCs to capture and process antigens. Processing of antigens by DCs involves binding, internalization, and display of antigenic determinants on the DC surface membrane. DCs carrying processed antigen travel to lymph organs where T lymphocytes recognize a complex on DCs consisting of major histocompatability complex (MHC)-peptide antigen determinate. DC contact activates T cells to secrete cytokines, which in turn stimulates B lymphocytes to divide and produce antibodies.

DCs have an extraordinary capacity to stimulate naive T cells and initiate primary immune responses (Liu et al., Nat Immunol. 2(7): 585-589, 2001). However, despite this capacity, DCs are very rare, and have been difficult to isolate in useful quantities in a cost efficient, time-efficient manner. One method to isolate blood DC involves several steps to deplete specific immune cell populations using resetting, adherence in cultures, and gradients to obtain a population of cells that primarily expresses (80-90%) the desired cell surface marker, CD11c. An alternative method to isolate DC from blood involves using antibodies attached to magnetic microbeads to deplete T cells, B cells, and Natural Killer cells and enriching for CD4 cells. The resulting cell population contains CDl lc+and CDI c- cell subsets.

However, the methods described above to isolate DC require several steps of manipulation, which compromises the investigators' ability to obtain consistent DC populations within and among laboratories. Thus, there remains a need to discover efficient, cost-effective methods for isolating dendritic cells.

### SUMMARY OF THE INVENTION

The invention provides efficient, cost-effect methods for isolating dendritic cells. Once isolated, dendritic cells are useful for stimulating an immune response (see, e.g., Schon et al., J. Reprod. Immunol. 50(2): 87-104, 2001).

Accordingly, in one aspect, the invention provides a method for isolating a population of dendritic cells and/or dendritic progenitor cells. The method includes contacting a population of cells with a plurality of FRIL family member molecules, and removing the unbound cells, wherein the cells bound to the FRIL family member molecules are an isolated population of dendritic cells and/or dendritic progenitor cells.

In certain embodiments of this aspect, the FRIL family member molecules are immobilized on a solid support. In some embodiments, the solid support is a magnetic bead. In some embodiments, the solid support is a tissue culture plate.

In some embodiments of this aspect, the plurality of FRIL family member molecules is labeled (e.g., detectably labeled). In certain embodiments, at least 70% of the isolated population of dendritic cells express CD11c. In certain embodiments, at least 70% of the isolated population of dendritic progenitor cells express CD11c. In certain embodiments, at least 78% of the isolated population of dendritic cells express CD11c.

In certain embodiments, the population of cells is selected from the group consisting of peripheral whole blood, peripheral blood mononuclear cells, umbilical cord blood, lymph node cells, lymphatic system cells, bone marrow cells, fetal liver cells, and spleen cells. In certain embodiments, the population of cells is from a human, a domesticated animal, or a laboratory animal. In some embodiments, the population of cell is pre-sorted to enrich the population for dendritic cells and/or dendritic progenitor cells.

In a further aspect, the invention provides an isolated population of dendritic cells and/or dendritic progenitor cells isolated by a method comprising contacting a population of cells with a plurality of FRIL family member molecules, and removing the unbound cells, wherein the cells bound to the FRIL family member molecules are an isolated population of dendritic cells and/or dendritic progenitor cells. In certain embodiments, the isolated population of dendritic cells and/or dendritic progenitor cells is from a human, a domesticated animal, or a laboratory animal.

In a further aspect, the invention provides a binding agent that specifically binds to a FRIL family member molecule. In certain embodiments, the binding agent is an antibody. In certain embodiments, the antibody is a monoclonal antibody or a polyclonal antibody. In some embodiments, the binding agent is labeled (e.g., detectably labeled).

### BRIEF DESCRIPTION OF THE DRAWINGS

FIGS. 1A-1C are schematic representations of flow cytometry analyses of a population of dendritic cells isolated according to the invention. FIG. 1A shows the relatively uniform size of the cells. FIG. 1B shows that the isolated cells are not bound by non-specific IgG-PE (phycoerythrin labeled IgG antibody). FIG. 1C shows that 78% of the cells are bound by a phycoerythrin labeled antibody that specifically binds to CD11c (CD11c-PE).

### DETAILED DESCRIPTION OF THE PREFERRED EMBODIMENTS

The invention provides a method for isolating and preserving dendritic cells and/or dendritic progenitor cells. Dendritic cells represent a heterogeneous and rare population (<1%) of cells produced in the bone marrow and distributed ubiquitously throughout the body of animals (including humans). Due to their derivation from either the mycloid or lymphoid lineage, dendritic cells have phenotypic and functional heterogeneity. This phenotypic and functional heterogeneity of dendritic cells is also distributed in the body in lymphatic ducts, peripheral blood, interstitial spaces of organs, lymphoid organs, and the epidermis.

All of the patents and publications cited herein reflect the knowledge in the art and are hereby incorporated by reference in entirety to the same extent as if each were specifically stated to be incorporated by reference. Any inconsistency between these patents and publications and the present disclosure shall be resolved in favor of the present disclosure.

In one aspect, the invention provides a method for isolating a population of dendritic cells and/or dendritic progenitor cells, comprising contacting a population of cells with a plurality of FRIL family member molecules, and separating the unbound cells, wherein the cells bound to the FRIL family member molecules are an isolated population of dendritic cells and/or dendritic progenitor cells.

By “dendritic cell” is meant a bone marrow-derived antigen presenting cell that, in some embodiments, has the phenotype and characteristics of the dendritic cells described in Steinman, Annu. Rev. Immunol. 9: 271-296, 1991. In some embodiments, a dendritic cell expresses MHC class II on its cell surface. In some embodiments, a dendritic cell of the invention has the phenotype and characteristics of the dendritic cells described in Santin et al., Obstet. Gynecol. 96(3): 422-430, 2000. In some embodiments, a dendritic cell of the invention has the phenotype and characteristics of the dendritic cells described in U.S. Pat. No. 6,017,527. In some embodiments, a dendritic cell of the invention has the phenotype and characteristics of the dendritic cells described in U.S. Pat. No. 6,274,378. In some embodiments, a dendritic cell of the invention has the phenotype and characteristics of the dendritic cells described in Turley et al., Science 288: 522-527, 2000. In particular embodiments, a dendritic cell of the invention expresses CD11c on its cell surface. In some embodiments, a dendritic cell of the invention expresses CD11b on its cell surface. In certain embodiments, a dendritic cell of the invention expresses CD83 on its cell surface. In some embodiments, a dendritic cell of the invention expresses CD32 on its cell surface. In particular embodiments, a dendritic cell of the invention expresses one or more of the following molecules on its cell surface: CD1a, CD4, and CD86.

By “dendritic progenitor cell” is meant a progenitor cell that will, upon becoming fully differentiated, develop into a dendritic cell (as defined above). As used herein, a “progenitor cell” refers to any normal somatic cell that has the capacity to generate fully differentiated, functional progeny by differentiation and proliferation. Progenitor cells include progenitors from any tissue or organ system, including, but not limited to, blood, mesenchymal, hair, embryonic, nerve, muscle, skin, gut (i.e., gastrointestinal), bone, kidney, liver, pancreas, thymus, and brain. In certain embodiments, the progenitor cell is a dendritic progenitor cell.

The term, “FRIL family” is used to mean a family of lectins, wherein each FRIL family member molecule preserves progenitor cells, and wherein each FRIL family member molecule binds to a normally glycosylated FLT3 receptor (see Moore et al., Biochim. Biophys. Acta 25027: 1-9, 2000). By “lectin” is meant a protein that binds sugar residues with high affinity. In accordance with the first aspect of the invention, the terms “bind,” “binds,” or “bound” are used interchangeably to mean that a FRIL family member molecule of the invention binds to a normally glycosylated FLT3 receptor with an affinity at least as high as or higher than the affinity with which the FLT3-Ligand binds the normally glycosylated FLT3 receptor. In some embodiments, a FRIL family member molecule binds to a normally glycosylated FLT3 receptor with an affinity that is at least as high as the affinity with which an antibody binds its specific ligand. In some embodiments, a FRIL family member molecule of the invention binds to a normally glycosylated FLT3 receptor with an affinity that is higher than the affinity with which an antibody binds its specific ligand. In some embodiments, a FRIL family member molecule of the invention binds to a normally glycosylated FLT3 receptor with a dissociation constant (K_{D}) of at least 10⁻⁷ M or 10⁻⁸ M, or 10⁻⁹ M, or at least 10⁻¹⁰ M, or a FRIL family member molecule of the invention binds to a normally glycosylated FLT3 receptor with a dissociation constant (K_{D}) of at least 10⁻¹¹ M. Standard methods for determining binding and binding affinity are known.

As used herein, by “preserves progenitor cells” is meant an ability of a FRIL family member (or mutant thereof or fusion protein comprising a FRIL family member or mutant thereof) to retain (i.e., preserve) progenitor cells in an undifferentiated state. The determination of a progenitor cell in an undifferentiated state can be determined using known assays (see, e.g., Kollet et al., Exp. Hematol. 28: 726-726, 2000; U.S. Pat. No. 6,084,060).

In accordance with the invention, by “normally glycosylated FLT3 receptor” is meant an FLT3 receptor that has a glycosylation pattern of an FLT3 receptor glycosylated by a normal cell. By “normal cell,” as used herein in accordance with all aspects of the present invention, is meant a cell that is not neoplastic. As used herein, by “neoplastic cell” is meant a cell that shows aberrant proliferation, particularly increased proliferation, that is not regulated by such factors as cell-cell contact inhibition and soluble regulators (e.g., cytokines or hormones), and that abnormally glycosylates the FLT3 receptor such that the glycosylation pattern on the FLT3 receptor on the neoplastic cells is abnormal and such that the FLT3 receptor on the neoplastic cell is not bound by a FRIL family member molecule.

By “FRIL family member” or “FRIL family member molecule” is meant one or more molecules of the FRIL family. In certain embodiments, a FRIL family member is from a legume, such as the garden pea or the common bean. Legumes are plants (“leguminous plants”) from a family (Leguminosae) of dicotyledonous herbs, shrubs, and trees bearing (nitrogen-fixing bacteria) nodules on their roots. In some embodiments, a FRIL family member is from members of the tribe Phaseoleae including, without limitation, Phaseolus vulgaris, Dolichos lab lab, Sphenostylis stenocarpa, Vigna sinensis, or Voandzeia subterranea. In some embodiments, a FRIL family member molecule is a mannose/glucose-specific legume lectin. (See Moore et al., Biochim. Biophys Acta 25027: 1-9, 2000; Colucci et al., Proc. Natl. Acad. Sci. USA 96: 646-650, 1999; Mo et al., Glycobiology 9: 173-179, 1999; and Hamelryck et al., J. Molec. Biol. 299: 875-883, 2000). In certain embodiments, the FRIL family member molecule that is isolated from a hyacinth bean (i.e., Dolichos lab lab) has an amino acid sequence which comprises the following eight amino acid sequence: TNNVLQXT (SEQ ID NO:11). A FRIL family member of the invention is, in some embodiments, encoded by a nucleic acid molecule comprising or consisting of the sequence of SEQ ID NO:1, SEQ ID NO:3, SEQ ID NO:5, or SEQ ID NO:7. In some embodiments, the FRIL family member molecule of the invention has an amino acid sequence comprising or consisting of the sequence of SEQ ID NO:2, SEQ ID NO:4, SEQ ID NO:6, SEQ ID NO:8, SEQ ID NO:9, or SEQ ID NO:10.

Other molecules falling into the definition of a FRIL family member molecule (e.g., mutants or fusion proteins), recombinant FRIL family member molecules, and methods for making and purifying such FRIL family member molecules (and methods for purifying nucleic acid molecules encoding such FRIL family member molecules) are described in U.S. Pat. No. 6,084,060; U.S. patent application Ser. No. 09/476,485 filed Dec. 30, 1999; Colucci et al., PCT Application No. PCT/US99/31307 (PCT Publication No. WO01/49851); and Colucci et al., PCT Application No. PCT/US98/13046 (PCT Publication No. WO98/59038), the entire disclosures of all of which are hereby incorporated by reference.

In some embodiments, each FRIL family member molecule binds to a cell surface molecule on a dendritic cell (or a dendritic progenitor cell) and has at least about 45% amino acid sequence identity with the amino acid sequence of another member of the FRIL family, or at least about 55% identity, or at least about 65% identity, or at least about 75% identity, or at least about 85% identity. In certain embodiments, each FRIL family member molecule binds to a cell surface molecule on a dendritic cell (or a dendritic progenitor cell) and has at least about 95% identity with the amino acid sequence of another member of the FRIL family (e.g., SEQ ID NO:2, SEQ ID NO:4, SEQ ID NO:6, SEQ ID NO:8, SEQ ID NO:9, or SEQ ID NO:10). Amino acid sequence identity and nucleic acid sequence identity between two proteins or two nucleic acid molecules can be measured according to standard methods (see, e.g., Pearson and Lipman, Proc. Natl. Acad. Sci. USA 85: 2444-2448, 1988; George, D. G. et al., in Macromolecular Sequencing and Synthesis, Selected Methods and Applications, pps. 127-149, Alan R. Liss, Inc. 1988; Feng and Doolittle, Journal of Molecular Evolution 25: 351-360, 1987; and Higgins and Sharp, CABIOS 5: 151-153, 1989; and the various BLAST programs of the National Center for Biotechnology, National Library of Medicine, Bethesda, Md.).

The invention stems from the discovery that an isolated population of dendritic cells and/or dendritic progenitor cells can be obtained by contacting a population suspected of containing dendritic cells and/or dendritic progenitor cells with a FRIL family member molecule, and retaining the cells bound to the FRIL family member molecule (i.e., the dendritic cells and/or dendritic progenitor cells). The cell surface molecule expressed on dendritic cells and/or dendritic progenitor cells to which a FRIL family member molecule binds may be a normally glycosylated FLT3 receptor, or it may be another molecule. Regardless of which cell surface molecule expressed on dendritic cells and/or dendritic progenitor cells a FRIL family member molecule binds, the invention provides a method for isolating dendritic cells and/or dendritic progenitor cells based on the ability of these cells to be bound by a FRIL family member molecule.

In some embodiments, the FRIL family member of the invention is purified. FRIL family member molecules are readily purified using standard techniques. Methods for purifying proteins are known in the art and include, without limitation, HPLC, SDS-PAGE, immunoprecipitation, recombinant protein production, affinity chromatography using specific antibodies, ion-exchange, size-exclusion, and hydrophobic interaction chromatography, or a combination of any of these methods. These and other suitable methods are described, e.g., in Marston, “The purification of eukaryotic proteins expressed in E. coli,” in DNA Cloning, Glover D. M., ed., Volume III, IRL Press Ltd., Oxford, 1987; Marston and Hartley, “Solubilization of protein aggregates,” pp. 266-267 in Guide to Protein Purification, Deutscher M. P., ed., Academic Press, San Diego, 1990; Laemmli, U. K., Nature 227: 680-685, 1970; Ausubel et al., Current Protocols in Molecular Biology, John Wiley & Sons, New York, N.Y., 1999; U.S. Pat. No. 6,084,060; and Gowda et al., J. Biol. Chem. 269: 18789-18793, 1994. A FRIL family member molecule can also be purified by binding to a mannose, which may be coupled on a sold support (e.g., a sepharose bead). Non-limiting sources from which naturally occurring FRIL family member molecules can be purified include Dolichos lab lab, Phaseolus vulgaris, Sphenostylis stenocarpa, Vigna sinensis, and Voandzeia subterranea.

Purification of a FRIL family member molecule from a legume is rapid and inexpensive, and results in a large amount of purified FRIL family molecule. FRIL family members are relatively abundant in legumes. For example, Dl-FRIL accounts for approximately 0.02% of the mass of hyacinth beans. By “purified” means a molecule, such as a protein (e.g., a FRIL family member molecule or a binding agent or antibody) or composition of that molecule, that is more free from other organic molecules (e.g., carbohydrates, nucleic acids, proteins, and lipids) that naturally occur with an impure molecule, and is substantially free as well of materials used during the purification process. For example, a protein is considered to be purified if it is at least approximately 60%, or at least approximately 75%, or approximately at least 85%, or approximately at least 90%, or approximately at least 95% pure, i.e., free from other organic molecules with which it naturally occurs and free from materials used during the purification process. A FRIL family member molecule can be easily purified from legumes, such as hyacinth beans (which can be grown pesticide-free), by mannose-affinity chromatography or ovalbumin affinity chromatography, and is more than 100 times cheaper to produce than recombinant cytokines.

The term “plurality of FRIL family member molecules” is used to mean one or more FRIL family member molecules. While the molecules may be identical (e.g., all of the FRIL family member molecules in the plurality of FRIL family member molecules are from Dolichos lab lab), the term also encompasses more than one identical FRIL family member molecule (e.g., two Dl-FRIL molecules, the FRIL family member molecule from Dolichos lab lab), or FRIL family member molecules from more than one source (e.g., a FRIL family member molecule from Dolichos lab lab and a FRIL family member molecule from Phaseolus vulgaris).

It is understood that when a population of cell is contacted with a plurality of FRIL family member molecules and those cells that bind to the FRIL family member molecules are isolated dendritic cells and/or dendritic progenitor cell according to the invention, not every FRIL family member molecule of the plurality of molecules necessarily binds to an isolated dendritic cell or dendritic progenitor cell. Rather, a surplus of FRIL family member molecules may be present in the plurality, such that not all FRIL family members will be bound but, rather, all dendritic cells and/or dendritic progenitor cells expressing a cell surface molecule that binds to a FRIL family member molecule will be bound by a FRIL family member molecule and thus isolated.

By “isolated” is meant a population of dendritic cells and/or dendritic progenitor cells that is separated from a larger population of cells, wherein the percentage of dendritic cells and/or dendritic progenitor cells in the isolated population is at least two fold greater than the percentage of dendritic cells and/or dendritic progenitor cells in the larger population. In some embodiments, the percentage of dendritic cells and/or dendritic progenitor cells in the isolated population is at least two fold greater than the percentage of dendritic cells and/or dendritic progenitor cells in the larger population. In some embodiments, the isolated population of dendritic cells and/or dendritic progenitor cells contains at least about 70% dendritic cells and/or dendritic progenitor cells, or at least about 75% dendritic cells and/or dendritic progenitor cells, or at least about 80% dendritic cells and/or dendritic progenitor cells, or at least about 90% dendritic cells and/or dendritic progenitor cells.

For example, when the population of cells contacted with the plurality of FRIL family member molecules is peripheral whole blood, the percentage of dendritic cells within whole blood may be, for example, 1%. The isolated population of dendritic cells and/or dendritic progenitor cells (i.e., isolated according to the methods of the invention) may contain, for example, at least about 70% dendritic cells and/or dendritic progenitor cells.

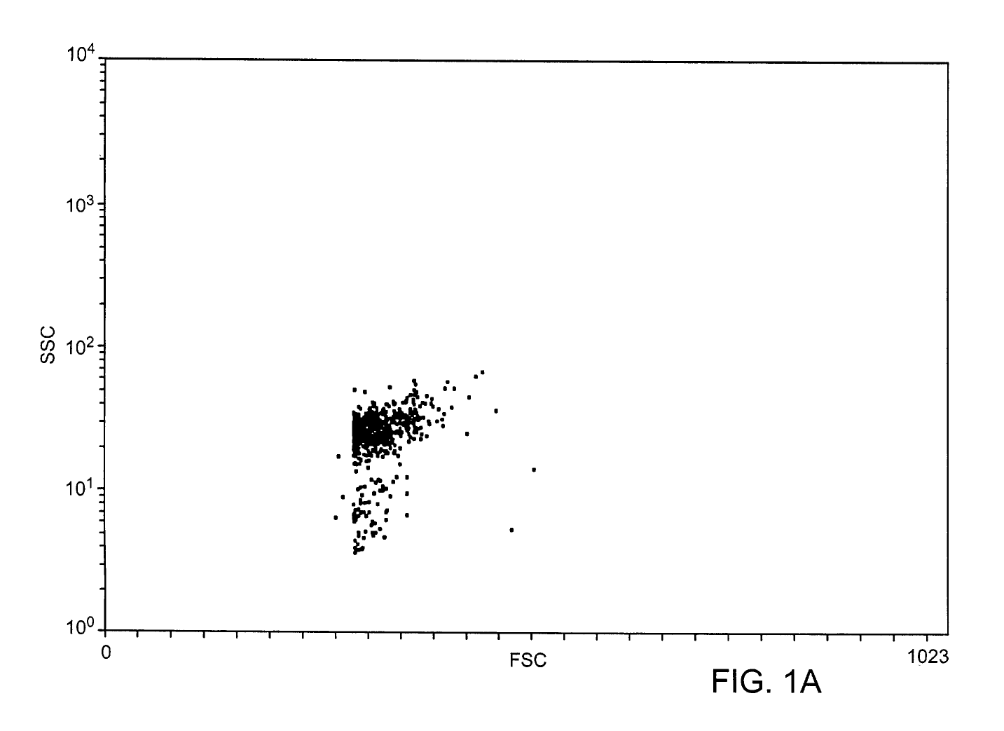

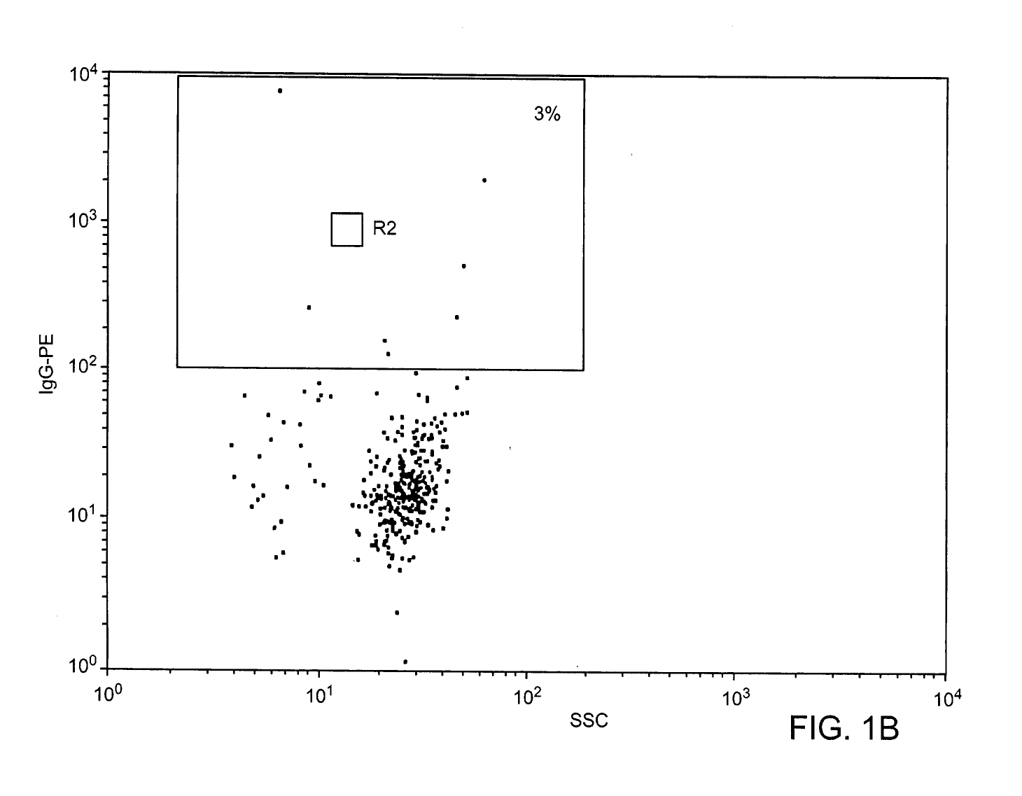

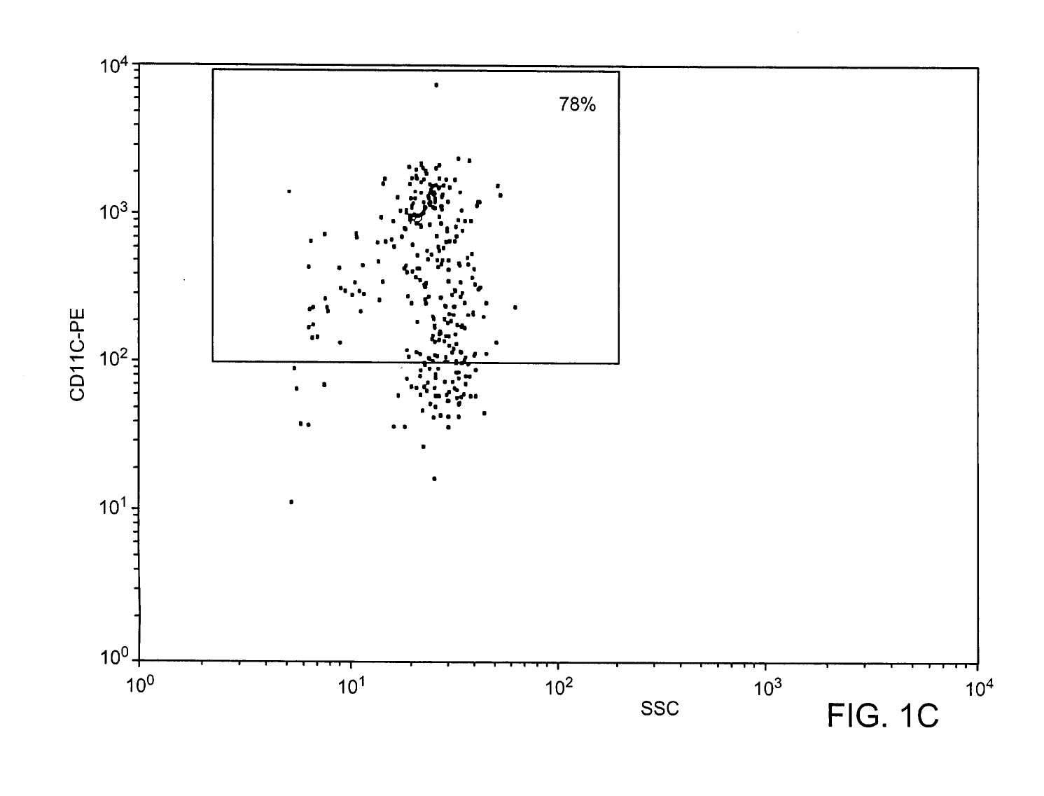

Dendritic cells and/or dendritic progenitor cells bind to FRIL family member molecules. Accordingly, a FRIL family member molecule of the invention provides an efficient and consistent method to enrich and isolate dendritic cells and/or dendritic progenitor cells. As described in the examples below, FRIL-loaded magnetic beads capture a rare population (0.05% -1%) of cells from adult peripheral blood. A majority of cells captured by FRIL-beads express CD11c on their cell surface (see FIGS. 1A-1C).

Dendritic cells and/or dendritic progenitor cells isolated according to the invention allows ex vivo expansion, manipulation, and re-infusion of specific antigen-loaded human dendritic cells and/or dendritic progenitor cells into a patient, which may enhance the immune system's ability to elicit a strong immune response against antigens that normally generate no immune response or a weak immune response. For example, tumor antigens often do not elicit strong immune responses. In addition, antigen-loaded dendritic cells and/or dendritic progenitor cells may be reinfused into patient to augment responses against the human immunodeficiency virus (HIV), or may be reinfused in vaccine preparations.

Ex vivo manipulation involves isolating and culturing dendritic progenitor cells in soluble mediators that induction proliferation and differentiation. For example, CD34-selected hematopoietic stem cells and progenitors cultured in granulocyte-macrophage colony stimulating factor (GM-CSF) and the ligand for the FLT3 tyrosine kinase receptor (FLT3-L) expand and differentiate dendritic progenitor cells to make mature dendritic cells (see, for example, U.S. Pat. No. 6,017,527). Pulsing ex vivo-generated dendritic cells with peptides associated with cancer can elicit a strong anti-tumor immune response. For example, pulsing dendritic cells with peptides associated with ovarian cancer cells activates cytotoxic CD8⁺ T cells to kill autologous tumor cells from women with advance ovarian cancer (Santin et al., Obstet. Gynecol. 96(3): 422-430, 2000).

In certain embodiments of the invention, the population of cells from which the dendritic cells and/or dendritic progenitor cells are purified is from an animal, including, without limitation, a domesticated animal, a laboratory animal or a human. By “domesticated animal” is meant an animal domesticated by humans, including, without limitation, a cat, dog, elephant, llama, horse, sheep, cow, pig, and goat. Also included as domesticated animals are non-mammals (e.g., turkeys and chickens). Non-limiting examples of a “laboratory animal” are non-human primates (e.g., chimpanzee, baboon), fish, frogs, worms, mice, rats, and rabbits.

The population of cells from which the population of dendritic cells and/or dendritic progenitor cells of the invention may be isolated includes any population of cells that contains cells derived from the bone marrow. Moreover, non-limiting sources of a population of cells of the invention include peripheral whole blood, peripheral blood mononuclear cells, umbilical cord blood, cells from lymph nodes (e.g., tonsils), cells from the lymphatic system, bone marrow cells, fetal liver cells, and spleen cells.

In certain embodiments of the invention, the FRIL family member molecules are labeled, such that purification of the FRIL family member molecule (and a cell to which the FRIL family member molecule is bound) can be accomplished. Numerous methods of labeling proteins are known in the art. The label can also be directly attached through a functional group on the FRIL family member. A FRIL family member molecule can be modified using standard techniques to contain a functional group. Some examples of suitable functional groups include, without limitation, amino, carboxyl, sulfhydryl, maleimide, isocyanate, isothiocyanate. One non-limiting label that may be incorporated into a FRIL family member molecule is biotin. Because biotin binds with high affinity to avidin, the biotin labeled FRIL family member molecule (and any cell to which the FRIL family member molecule is bound) can be purified, for example, on avidin (or streptavidin) -coated magnetic beads.

In a further aspect, the invention provides a binding agent, such as an antibody, that specifically binds to a FRIL family member molecule. By “specifically binds” is meant that a binding agent of the invention recognizes and binds to a FRIL family member of the invention with a dissociation constant (K_{D}) of at least 10⁻⁵ M, or at least 10⁻⁶ M, or at least 10⁻⁷ M, or at least 10^{31 8} M, or, a binding agent of the invention binds to a FRIL family member with a dissociation constant (K_{D}) of at least 10⁻¹⁰ M under physiological conditions, in a physiologically acceptable solution, or under conditions which approximate physiological conditions with respect to ionic strength, e.g., 140 mM NaCl, 5 mM MgCl₂. By “physiologically acceptable solution” is meant an inert solution, such as sterile saline solution or tissue culture medium, which is non-toxic to the cells. Non-limiting physiologically acceptable solutions are those comprising 140 mM NaCl or 5 mM MgCl₂. Standard methods for determining binding and binding affinity are known.

A binding agent, in accordance with the invention, need not be any particular size or have any particular structure so long as it specifically binds to a FRIL family member molecule. Non-limiting examples of binding agents include small molecules, chemicals, peptides, and proteins, such as hormones and antibodies.

In some embodiments, where the binding agent is an antibody, the antibody is purified. Antibodies of the invention that specifically bind to a FRIL family member molecule may be, without limitation, a monoclonal antibody, a chimeric antibody, a humanized antibody, a genetically engineered antibody, a bispecific antibody (where one of the specificities of the bispecific antibody specifically binds to a FRIL family member molecule), antibody fragments (including but not limited to “Fv,” “F(Ab)_{2,}” “F(Ab),” and “Dab”); and single chains representing the reactive portion of an antibody (“SC-MAb”). Methods for making antibodies and other binding agents are well known (see, e.g., Coligan et al., Current Protocols in Immunology, John Wiley and Sons, New York City, N.Y., 1991; Jones et al., Nature 321: 522-525, 1986; Marx, Science 229: 455-456, 1985; Rodwell, Nature 342: 99-100, 1989; Clackson, Br. J. Rheumatol. 3052: 36-39, 1991; Reichman et al., Nature 332: 323-327, 1988; Verhoeyen, et al., Science 239: 1534-1536,1988).

In a non-limiting example of a method for isolating a dendritic cell using an antibody that specifically binds to a FRIL family member, a population of cells is contacted with the plurality of FRIL family members, and secondarily contacted with anti-FRIL antibodies (i.e., antibodies that specifically bind to a FRIL family member). The population of cells can then be contacted with, for example, protein A immobilized on a solid support, and the unbound cells rinsed off a physiologically acceptable solution. In this method, the isolated population of dendritic cells remains bound to the solid support. The term “solid support” includes any surface, including, without limitation, the surface of a sepharose bead, a gel, a matrix, a magnetic bead, or a plastic surface (e.g., the bottom of a tissue culture dish or flask). In certain embodiments, the solid support is the bottom of a tissue culture plate, and the unbound cells are separated by rinsing the tissue culture plate with a physiologically acceptable solution.

In addition, if the anti-FRIL antibody is from a particular animal (e.g., the anti-FRIL antibody is a murine antibody), FRIL-bound cells can be purified by contact with, for example, goat anti-mouse antibodies immobilized on a solid support (e.g., magnetic beads). The dendritic cells that are FRIL-bound can then be purified by contacting population of cells with a magnet and rinsing away the unbound cells.

In certain embodiments of the invention, the plurality FRIL family member molecules are themselves immobilized on a solid support. Where the solid support is a magnetic bead, the unbound cells are separated by applying a magnet to the population of cells contacted with the FRIL family member molecules immobilized on the magnetic bead and rinsing off the unbound cells. Methods for isolating cells that bind FRIL family member molecule-coated magnetic beads are described in the examples below. Magnetic beads are commercially available (e.g., from Dynabeads, Lake Success, N.Y. (for, e.g., tosylactivated beads which allow for direct binding of FRIL to the bead); or from Miltenyi Biotec, Auburn, Calif. (for, e.g., streptavidin-coated beads)). The FRIL family member molecule can be conjugated to a magnetic bead by known methods (e.g., via amino- or sulfhydryl-groups of the FRIL family member molecule).

In some embodiments, the magnetic beads on which either FRIL or binding agents that specifically bind to a FRIL family member molecule (e.g., FRIL-specific antibodies) are immobilized are the MACS super-paramagnetic MicroBeads (from Miltenyi Biotec) which are extremely small, approximately 50 nm in diameter (MACS beads are about one million times smaller in volume than eukaryotic cells). MACS beads react like magnetic antibodies; thus, magnetic labeling is achieved within minutes. MACS MicroBeads form a stable colloidal suspension and do not precipitate or aggregate in magnetic fields. MACS MicroBeads biodegradable, so that cells bound to MACS MicroBeads are able to retain their physiological function. This property of MACS beads is particularly useful for bead-sorted FRIL family member-binding cells, which bind the FRIL family member with such high affinity that it is difficult to remove the beads.

It will be understood, however, that in all of the methods of the invention wherein a dendritic cell and/or dendritic progenitor cell expressing a cell surface molecule that binds to a FRIL family member molecule is attached to a solid surface (via binding to the FRIL family member on the solid support), the dendritic cell and/or dendritic progenitor cell can be readily removed from the solid surface by standard methods. In one non-limiting example of such a method, a surplus of free FRIL family member molecules is added to the solid surface-bound cells, thereby competing the dendritic cells and/or dendritic progenitor cells off of the solid support with the free FRIL family member molecule.

FRIL family member molecules may be directly or indirectly attached to the bottom of a tissue culture plate. Following a standard “panning” protocol (see, e.g., Stengelin et al., EMBO J. 7(4): 1053-1059, 1988; Aruffo and Seed, Proc. Natl. Acad. Sci. USA 84(23): 8573-8577, 1987), a population of cells suspected of containing dendritic cells and/or dendritic progenitor cells is incubated on the plate such that cells expressing a cell surface molecule that binds to a FRIL family member molecule will be bound by the immobilized FRIL family member molecule. The plate is then gently rinsed with a physiologically acceptable solution, thereby removing the unbound cells while leaving the FRIL family member-binding population of cells attached to the FRIL family member-coated plate.

In certain embodiments, the FRIL family member molecule or binding agent that specifically binds to the FRIL family member molecule is detectably labeled. By “detectably labeled” is meant that the FRIL family member or binding agent is attached to a label that is detectable visually or instrumentally. For example, a chromophoric or fluorogenic molecule can be conjugated to the FRIL family member molecules or binding agent by means of coupling agents, such as dialdehydes, carbodiimides, and dimaleimides. Non-limiting detectable labels include phycoerythrin and FITC. Accordingly, when population of cells is contacted with a detectably labeled FRIL family member molecule (or with the FRIL family member molecule followed by a detectably labeled binding agent, such as an antibody, that specifically binds to the FRIL family member molecule), bound cells can be isolated by cell sorting on a flow cytometry instrument.

In certain embodiments of the invention, the population of cells from which the dendritic cells and/or dendritic progenitor cells are isolated is a population of cells that is enriched for dendritic cells and/or dendritic progenitor cells. For example, the dendritic cell and/or dendritic progenitor cell-enriched population may be a sorted population, wherein a cell of the sorted population does not express a T cell receptor and/or a B cell receptor. Following this negative sort (i.e., a sort, wherein the cells retained do not express a T cell receptor and/or a B cell receptor), the sorted population is positively sorted for an ability to bind to a FRIL family member.

In one example, the population of cells is first enriched for dendritic cells and/or dendritic progenitor cells by sorting the population by flow cytometry or by magnetic bead selection. Thus, a population of cells (e.g., human umbilical cord blood cells) may be first contacted with chromophore-labeled antibodies (or other molecule such as a ligand) which specifically bind the T cell receptor. Following binding, the population of cells is then negatively sorted by flow cytometry, where the cells which are not bound by the antibodies (and so do not express the T cell receptor) are retained and further sorted for an ability to bind a FRIL family member, wherein the population of cells that binds a FRIL family member is an isolated population of dendritic cells and/or dendritic progenitor cells according to the invention.

A population of cells may also be enriched for dendritic cells and/or dendritic progenitor cells by contacting the population with a molecule, such as an antibody, which specifically binds a B cell receptor, wherein the molecule is attached to a solid support, such as a magnetic bead. The population is then negatively sorted by applying a magnet to the beads, and retaining the cells that do not bind the beads and so are not attracted to the magnet. These sorted cells are then further sorted for an ability to bind a FRIL family member, wherein the population of cells that binds a FRIL family member is an isolated population of dendritic cells and/or dendritic progenitor cells according to the invention.

In another aspect, invention provides an isolated population of dendritic cells and/or dendritic progenitor cells isolated by a method comprising contacting a population of cells with a plurality of FRIL family member molecules, and separating the unbound cells, wherein the cells bound to the FRIL family member are an isolated population of dendritic cells and/or dendritic progenitor cells. “Isolated”, “FRIL family member molecule,” “progenitor cell,” and “bound” are as described above.

In certain embodiments, the isolated population of progenitor cells is from a human or a domesticated animal.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLE 1

Enrichment of Peripheral Blood Mononuclear Cells

The following standard procedure was used to prepare peripheral blood mononuclear cells (PBMCs) from either umbilical cord blood or peripheral blood and to remove red blood cells. (Human umbilical cord blood (CB) samples were obtained from full term deliveries.) This procedure was always performed under sterile conditions (e.g., in a tissue culture hood). This general protocol has been described (see, e.g., Rubinstein et al., Proc. Natl. Acad. Sci. 92: 10119-10122, 1995; Clin. Lab. Haem. 20: 341-343, 1998; Vox Sang. 76: 237-240, 1999)

Blood was collected under sterile condition (100 μl are saved for cell count) and the total volume of blood was recorded. The blood was collected into tubes containing an appropriate amount of the anti-coagulant, Citrate-Phosphate-Dextrose-Adenine (commercially available from Sigma Chemical Co., St. Louis Mo.; Catalog No. C4431, 50 ml). A stock of Citrate-Phosphate-Dextrose-Adenine was made of 5 ml in 10 cc syringes.

Next, hetastarch (stock is 6% in 0.9% NaCl, commercially available from Abbott, NDC 0074-7248-03, 500 ml) was added to blood to a 1.2% final concentration, and mixed well. The blood was next allowed to sit at room temperature (approximately 25° C.) for 45 minutes. The color and clarity of the top layer was noted during this time.

During this 45 minute “sitting time,” an initial cell count was made (using a haemocytometer). To do this, 180 μl of 2% acetic acid was mixed with 20 μl of the saved cells (leaving 80 μl) for a 1:10 dilution, and the cells were counted.

After the 45 minute “sitting time”, the leukocyte-enriched top layer was transferred to a new 50 ml conical tube, and the leukocytes washed twice with degassed HAEM. (HAEM is HBSS (Invitrogen Life Technologies, Carlsbad, Calif.; Catalog No. 14025076) +0.1% AIM V Media (Invitrogen Life Technologies, Catalog No. 12055083) +2 mM EDTA (Sigma, Catalog No. E-7889, Lot # 110K89271), and is degassed by shaking for five minutes.) To wash, the volume of the cells was brought up to 40 ml with degassed HAEM, then the cells were centrifuged for 10 minutes at 400 xg at 11° C. in a Beckman GS-6R centrifuge, and the supernatant aspirated.

After washing, the cell pellet was resuspended in I mL HAEM, and the remaining red blood cells lysed. To lyse the red blood cells, 9 nL of NH₄Cl solution (0.72% NH₄Cl, 10 mM EDTA, StemCell Technologies, Inc., Vancouver, Canada) was added to 1 ml of cell suspension, the tube mixed and placed on ice for five minutes.

Next, the cells were washed twice in degassed HAEM (volume was raised to 40 ml with degassed HAEM, the cells centrifuged for 5 min at 1300 RPM and 11° C., then the supernatant was aspirated). The cell pellet was then resuspended in 20 ml degassed HAEM. Finally, the remaining cells were counted, and were ready to be used.

### EXAMPLE 2

Methods to Isolate Dendritic Cells from Non-Blood Tissues

The following methods are used to obtain single cell suspensions from non-blood tissues, from which FRIL-binding dendritic cells can be isolated according to Examples 3-6. This procedure was always performed under sterile conditions (e.g., in a tissue culture hood).

Tonsil Tissue

Tonsil tissue is cut into small pieces, and the pieces are digested by placing the tissue in media containing with collagenase and Dnasel (both commercially available from Sigma Chemical Co.). From this digestion, the free (i.e., single) cells are layered on a 50% Percoll gradient according to standard methods (Percoll commercially available from AP Biotech). Standard cell biology methods are known (see, e.g., Ausubel et al., Current Protocols in Molecular Biology, John Wiley & Sons, New York, N.Y., 1999; Coligan et al., Current Protocols in Immunology, John Wiley & Sons, New York, N.Y., 1994). The low density cells are collected from the Percoll gradient, and these cells are now ready for FRIL cell selection (see Examples 3, 4, 5, or 6).

Skin

Skin is obtained from patients undergoing reconstructive surgery (e.g., breast or abdomen). The tissue is incubated in low concentrations of trypsin to generate single cell suspensions. This single cell suspension is now ready for FRIL cell selection.

Lymph Node

Lymph node tissue is treated with Collagenase in Ca²⁺⁺-free medium to generate single cell suspensions. This single cell suspension is now ready for FRIL cell selection.

Spleen

Spleen tissue is treated with Collagenase in Ca²⁺⁺-free medium to generate single cell suspensions. Next, the single cells are layered on a 50% Percoll gradient according to standard methods. The low density cell fraction is collected from the Percoll gradient, and these cells are now ready for FRIL cell selection.

### EXAMPLE 3

FRIL Cell Selection to Isolate Dendritic Cells

The following standard procedure was used to isolate dendritic cells from the peripheral blood mononuclear cells (PBMCs) prepared as described in Example 1. This procedure was always performed under sterile conditions (e.g., in a tissue culture hood).

First, biotinylated FRIL (bFRIL) was prepared. To do this, FRIL was purified according to standard methods (see U.S. Pat. No. 6,084,060; Mo et al., Glycobiology 2: 173-179, 1999; Kollet et al., Exp. Hematol. 28: 726-726, 2000; Hamelryck et al., J. Molec. Biol. 299: 875-883, 2000. Next, purified FRIL was biotinylated according to Pierce Chemical Co. protocol (Pierce Biotechnology, Rockford, Ill.), and kept at a stock of 1 ug/ml.

The leukocyte-rich PBMCs from Example 1 were centrifuged for 5 minutes at 1300 RPM at 11° C. (cells were in 20 ml from the protocol described in Example 1. Next, the supernatant was aspirated, and the cells resuspended to 500 ul with 450 ul degassed HAEM (this assumes there are 50 ul of cells and excess HAEM).

Next, the cells are prepared with bFRIL. To do this, 50 ul of bFRIL (stock is 1 ug/ml) was added to the 500 ul of cells and mixed. This created a 1:10 dilution bFRIL to volume of cells. The bFRIL plus cells mixture were next place on ice for 30 minutes. Next, the volume of the bFRIL plus cells mixture was raised to 40 mls with degassed HAEM, and the mixture was centrifuged 5 minutes at 1300 RPM at 11° C. The supernatant was aspirated and the cells were resuspended in 500 ul of degassed HAEM as before.

Next, the cells were prepared with Strepavidin (SA) beads (commercially available from Miltenyi Biotec Auburn, Calif.; Catalog No. 100-18-101). The SA beads were added to the 500 ul of cells such that the SA Beads are concentrated at 5 ul beads per 107 cells. The cells are mixed and placed on ice for 5 minutes. Next, the volume was raised to 2 ml with 1.5 ml degassed HAEM.

Meanwhile, a column was prepared for cell selection. To do this, a MACS LS⁺ separation column (Miltenyi Biotec, Catalog No. 424-01) with a pre-separation filter (“pre-filter”; 30 um; Miltenyi Biotec, Catalog No. 414-07) was placed in the magnet (MidiMACS separation apparatus; Miltenyi Biotec, Catalog No. 423) with a 50 ml conical test tube placed below column. Three ml degassed HAEM were run through the pre-filter to start collecting the “FRIL negative cells”.

Immediately after preparing the column (i.e., while there was still a meniscus of HAEM in the column), the cell sample (i.e., cells with bFRIL plus SA beads) were transferred from the test tube to the pre-filter. Occasionally, the pre-filter was jostled slightly to release a vacuum created. Care was taken in avoid splashing the cells too far up on the column wall. After the sample was run through the column, 2 ml degassed HAEM was pipetted into the conical test tube where the cells were kept prior to being run through the column to wash the test tube and collect the excess cells. Then, the 2 ml was pipetted into the prefilter and allowed to run through the column. Next, 2 mls of degassed HAEM were run through the pre-filter before the column ran dry from the last wash.

After the pre-filter had no more drops, the pre-filter was jostled and then removed. Next, the column was washed with a series of washes. First, three washes were performed by adding 2 mls of degassed HAEM to the column and allowing this to run through the column. Next, two washes are performed by adding 5 mls of degassed HAEM to the column and allowing this to run through the column. Care was taken never to allow the column to become dry. In other words the tip of the column was always dripping.

After the washes, the “FRIL positive cells” were collected. To do this, the column was removed from the magnet and placed on top of a sterile 15 ml conical test tube labeled “FRIL pos cells” which was positioned directly beneath the tip of the column, so that the tip was inside of the test tube. This was done so that the cells would be caught in the tube if they splattered when the column was removed from the magnet.

Immediately after removing the column, 2 ml of degassed HAEM was pipeted (i.e., added) into the column, and the HAEM was immediately plunged forcefully through the column into the test tube with the sterile plunger provided with the column. The final volume containing the cells was between 2 ml and 2.5 ml.

The plunged cells were centrifuged for five minutes at 1300 RPM at 11° C., and the supernatant was discarded. The cell pellet was then resuspended in the 2 mls of fresh HAEM (mixed using a pipet).

Next, the FRIL positive cells were counted. To do this, a 1:100 dilution of cells was made by mixing 180 ul of 1:100 Trypan Blue Stain (stock 0.4%; Sigma Chemical Co., Catalog No. T-81 54) with 20 ul of the “FRIL pos cells” for a 1:10 dilution. 20 ul of this 200 ul 1:10 dilution was next mixed with 180 ul of 1:100 Trypan Blue stain for the 1:100 dilution. 20 ul of the 200 ul 1:10 dilution was pipetted into each well of one row consisting of 6 wells in a 96 microwell plate (Nunclon™Δ; commercially available from Nunc, Rochester, N.Y.; Catalog No. 136528). This was repeated for the 1:100 dilution of cells in the next row of the 96 microwell plate. Three wells of the 1:10 dilution were immediately counted for a viability check by counting the alive and dead cells after a minute of being in the presence Trypan blue (living cells exclude Trypan blue). For the viability count, the number of alive cells was divided by the total number of alive and dead cells, and then multiplied by 100 for a percentage.

Next, 15 minutes after making the 96 microwell plate, each of the 6 wells of the 1:100 dilution were counted for alive and dead cells as one total cell count. To calculate the number of cells and recoveries, the average of the number of cells was divided by 0.02 ml for the concentration in each well. This number was then multiplied by 100 for the dilution factor. This number was then multiplied by the total volume of the plunged cells. For the recover, the final cells count was divided by the initial number of cells and multiplied by 100 for a percentage. Now, the FRIL positive cells were ready to be used.

To determine the CD11 cell surface expression on FRIL positive cells, cells were pelleted (by centrifugation) in FACS tubes (5 ml polycarbonate round bottom tubes (VWR, West Chester, Pa.; Catalog No. 352063), and the media removed by aspiration. Next, 20 ul of the following antibodies were added to each tube: Mouse IgG₁-PE Isotype control (BioSource International, Camarillo, Calif.; Catalog No. AML2317) and mouse anti-human CD11c-PE (BioSource International, Catalog No. AHS1157). The cells were resuspended in the antibody, and the tubes covered with foil to block light, and then incubated on ice for 30 minutes. Next, the cells were washed with HBSS without phenol red (commercially available from Invitrogen, Carlsbad) (i.e., by filling the tube with HBSS, pelletting the cells by centrifugation, and aspirating the supernatant. The cell pellet was next resuspended in HBSS without phenol red by adding HBSS to 0.5 ml total volume. Next, 20 ul of Propidium Iodide (PI) (50 ug/ml stock, freshly thawed; commercially available from Sigma Chemical Co., St. Louis, Mo.) was added to the cells and mixed. The cells were then analyzed by flow cytometry analysis on a Becton Dickson FACScan (Becton Dickson, Franklin Lakes, N.J.).

Using the procedures outlined in this example, from a starting population of 3×10⁸ peripheral blood mononuclear cells, approximately 1.6×10⁵ dendritic cells were isolated.

As shown in FIG. 1, which shows the results of one representative experiment, approximately 78% of the isolated dendritic cells were positive for expression of the CD11c cell surface antigen.

### EXAMPLE 4

Use of Magnetic Beads Coated with a FRIL Family Member to Isolate Dendritic Cells

Using magnetic beads coated with a non-limiting FRIL family member, Dl-FRIL, a population of dendritic cells was isolated. To do this, the following methods were used.

Preparation of FRIL-Beads for Cell Isolation

Dl-FRIL was purified from Dolichos lab lab seeds according to standard methods (see U.S. Pat. No. 6,084,060; Mo et al., Glycobiology 9: 173-179, 1999; Kollet et al., Exp. Hematol. 28: 726-726, 2000; Hamelryck et al., J. Molec. Biol. 299: 875-883, 2000. Dl-FRIL can be immobilized on magnetic beads (M-280 Dynabeads Tosylactivated; Dynal, Lake Success, N.Y.) via amino- and sulfhydryl-groups of the lectin according to the manufacturer's directions. Dl-FRIL can also immobilized on magnetic beads by a biotin-strepavidin interaction.

In this example, Dl-FRIL was immobilized on magnetic beads by a biotin-strepavidin interaction. Biotinylation of Dl-FRIL via primary amine-groups (EZ-LinkJ Sulfo-NHS-LC-LC-Biotin, Pierce Chemical Company, Rockford, Ill.) was carried out according to the manufacturer's directions. Biotinylated Dl-FRIL was incubated with strepavidin-labeled magnetic beads (Dynal or Miltenyi Biotec, Auburn, Calif.) according to the manufacturer's directions.

Umbilical cord blood mononuclear cells (CBmnc) were prepared as described in Example 1.

Dl-FRIL-Bead Cell Isolation

Dl-FRIL-coated beads specifically bound a minor mnc population found in CB, peripheral blood, and bone marrow. A ten-fold excess of Dl-FRIL-beads was incubated with the cells. For CB, where Dl-FRIL-beads captured approximately 1% of mnc, the ratio of beads to cells was 1:10, or 10-fold greater number of beads for every target cell.

Dl-FRIL-beads were washed twice in serum-defined medium prior to use. An aliquot of Dl-FRIL-beads was added to 10 mL of serum-defined medium in a 15 mL conical centrifuge tube (Falcon, Becton-Dickinson, Lincoln, N.J.), mixed, and placed in a magnet (Dynal or Miltenyi Biotec, depending on source of magnetic beads) for ten minutes. Medium was aspirated with a 10 mL pipette without disturbing beads bound to walls of centrifuge tube by the magnet charge from the magnet. After washing, 0.5 mL of serum-defined medium was added to the tubes to wash the beads from the walls to the bottom of the conical tube. Medium was added to beads in a small volume (<2 mL) and the centrifuge tube was tumbled on a rocker in a cold room (i.e., at approximately 4° C.) for one hour. After incubation, serum-defined medium was added to a final volume of 10 mL and the tube was placed in the magnet for ten minutes. Medium was removed by aspiration without disturbing cells bound to Dl-FRIL-beads on the walls of the centrifuge tube via the magnetic charge. Cells were washed a second time by removing the conical tube from the magnet, adding 10 mL of serum-defined medium, mixing cells, and placing the conical tube back onto the magnet. Following aspiration of the medium, the final volume was adjusted to 2 mL.

Cell Surface Phenotypic Properties of Dl-FRIL Bead-Selected CB mnc

The cell surface phenotypic properties of Dl-FRIL bead-selected CB mnc was characterized by flow cytometry. Table 1 shows the phenotypes (by percentage of cells expressing the indicated cell surface phenotype marker) of the three CB cell populations: (1) cells not selected by Dl-FRIL-beads (Dl-FRIL-); (2) cells that detached from Dl-FRIL-beads after overnight incubation in the cold room (i.e., 4° C.) on a rocker (Dl-FRIL+); and (3) cells that retained Dl-FRIL-beads after overnight incubation (Dl-FRIL++). The two Dl-FRIL-binding cell populations were analyzed separately to see whether tightness of binding (avidity) related to type of cells selected. Isotype control levels were set at 2%; all values of 2% represent no reactivity with test antibody.

TABLE 1
Flow cytometric analysis of DI-FRIL-selected CB mnc

| | | DI-FRIL⁻ | DI-FRIL⁺ | DI-FRIL⁺⁺ |
|---|---|---|---|---|
| Antigen | Cell Type | (%) | (%) | (%) |
| CD3 | Mature T | 26 | 35 | 6 |
| CD11b | Mac-1, CR3 | 19 | 35 | 67 |
| CD11c | LeuCAMc | 10 | 22 | 32 |
| CD13 | Pan myeloid, CFU-GM | 5 | <2 | <2 |
| CD19 | Pan B | 4 | 5 | 12 |
| CD32 | Low affinity IgG Fcγ-R | 5 | 19 | 26 |
| CD33 | Myeloid progenitors | 3 | 2 | 8 |
| CD34 | Pan progenitors | <2 | <2 | <2 |
| CD38 | Activated T | 88 | 96 | 93 |
| CD42a | Platelet gpIX | 5 | 2 | 7 |
| CD69 | Early activation ag (EA-1) | 6 | 8 | 14 |
| CDw90 | Thy-1, progenitor subset | 8 | 14 | 13 |
| CD117 | c-kit, progenitors | 4 | 2 | 2 |

Cells expressing dendritic cell (DC) markers, CD11b and CD11c, were enriched approximately 2-fold in the D1-FRIL⁺ cell population and over 3-fold in the D1-FRIL++ cell population (Table 1). The rare hematopoietic dendritic cell population is useful in inducing tumor regression and for the treatment of AIDS.

### EXAMPLE 5

Use of Plastic Tissue Culture Plates Coated With a FRIL Family Member to Isolate Dendritic Cells and/or Dendritic Progenitor Cells

In this example, dendritic cells and/or dendritic progenitor cells are purified by “panning” a population of cells suspected of containing dendritic cells and/or dendritic progenitor cells on plastic petri plates onto which are absorbed a FRIL family member. This protocol follows, in general, the protocol described in e.g., Stengelin et al., EMBO J. 7(4): 1053-1059, 1988; Aruffo and Seed, Proc. Natl. Acad. Sci. USA 84(23): 8573-8577, 1987.

To do this, Pv-FRIL is purified from Phaseolus vulgaris according to standard methods (see, e.g., Colucci et al., PCT Publication No. WO01/49851). The purified Pv-FRIL resupended in HBSS and allowed to absorb onto bacterial (i.e., non-tissue culture treated) plastic petri plates under sterile conditions. After the Pv-FRIL has absorbed onto the plastic (e.g., after incubation for two hours at room temperature), the Pv-FRIL solution is removed, and the plate is blocked with a solution of bovine serum albumin.

Next, the population of cells prepared as described in Examples 1 or 2 are added to the petri dish and allowed to incubate on the dish. One non-limiting incubation is at 37° C. for 30 minutes. Next, the petri plate is tilted and the unbound cells allowed to slide off the plate. The unbound cells are removed by aspiration. The petri dish is next gently washed with sterile HBSS and, upon tilting the petri plate, the unbound cells are allowed to slide off the plate and are removed by aspiration.

The cells that remain bound to the plate are an isolated population of dendritic cells and/or dendritic progenitor cells of the invention.

### EXAMPLE 6

Cell Sorting to Isolate Dendritic Cells and/or Dendritic Progenitor Cells

Biotinylated Dl-FRIL is prepared as described in Example 4.

A population of cells suspected of containing dendritic cells and/or dendritic progenitor cells is incubated under sterile on ice with biotinylated Dl-FRIL (e.g., for about 30-60 minutes). The cells are washed with HBSS, and FITC-labeled streptavidin is added. The cells are allowed to incubate on ice. The cells are next washed with HBSS and resuspended in sterile HBSS.

A control group of cells is stained with only the FITC-labeled streptavidin.

Next, the cells are run on a flow cytometer (commercially available from, e.g., Becton-Dickson). The cells that stain positive for the FITC label are collected. These FITC-positive cells are an isolated population of dendritic cells and/or dendritic progenitor cells of the invention.

1. A method for isolating a population of dendritic cells and/or dendritic progenitor cells, comprising contacting a population of cells with a plurality of FRIL family member molecules, and removing the unbound cells, wherein the cells bound to the FRIL family member molecules are an isolated population of dendritic cells and/or dendritic progenitor cells.

2. The method of claim 1, wherein the plurality of FRIL family member molecules is immobilized on a solid support.

3. The method of claim 2, wherein the solid support is a magnetic bead.

4. The method of claim 2, wherein the solid support is a tissue culture plate.

5. The method of claim 1, wherein the plurality of FRIL family member molecules is labeled.

6. The method of claim 1, wherein the plurality of FRIL family member molecules is detectably labeled.

7. The method of claim 1, wherein at least about 70% of the isolated population of dendritic cells and/or dendritic progenitor cells express CD11c.

8. The method of claim 1, wherein at least about 78% of the isolated population of dendritic cells and/or dendritic progenitor cells express CD11c.

9. The method of claim 1, wherein the population of cells is selected from the group consisting of peripheral whole blood, peripheral blood mononuclear cells, umbilical cord blood, lymph node cells, lymphatic system cells, bone marrow cells, fetal liver cells, and spleen cells.

10. The method of claim 1, wherein the population of cells is from a human.

11. The method of claim 1, wherein the population of cells is from the group consisting of a domesticated animal and a laboratory animal.

12. The method of claim 1, wherein the population of cell is pre-sorted to enrich the population for dendritic cells and/or dendritic progenitor cells.

13. An isolated population of dendritic cells and/or dendritic progenitor cells isolated by contacting a population of cells with a plurality of FRIL family member molecules and removing the unbound cells.

14. The isolated population of claim 13, wherein at least about 70% of the isolated population of dendritic cells and/or dendritic progenitor cells express CD11c.

15. The isolated population of claim 13, wherein at least about 78% of the isolated population of dendritic cells and/or dendritic progenitor cells express CD11c.

16. The isolated population of claim 13, wherein the population of cells is selected from the group consisting of peripheral whole blood, peripheral blood mononuclear cells, umbilical cord blood, lymph node cells, lymphatic system cells, bone marrow cells, fetal liver cells, and spleen cells.

17. The isolated population of claim 13, wherein the population of cells is from a human.

18. A binding agent that specifically binds to a FRIL family member molecule.

19. The binding agent of claim 12, wherein the binding agent is an antibody.

20. The binding agent of claim 12, wherein the binding agent is labeled.
